(19) United States
(12) Patent Application Publication
(10) Pub. No.: US 2010/0022989 A1
(43) Pub. Date: Jan. 28, 2010

(54) Steerable catheter and method of making the same

(76) Inventors: Ronald S. Parasmo, North Palm Beach, FL (US); Michael A. Renick, Lake Park, FL (US)

Correspondence Address: SIMPSON & SIMPSON, PLLC, 5555 MAIN STREET, WILLIAMSVILLE, NY, 14221-5406, US

(21) Appl. No.: 12/220,621

(22) Filed: Jul. 25, 2008

(51) Int. Cl. A61M 25/01; C21D 1/40

(52) U.S. Cl. 604/528; 219/162

(57) ABSTRACT

A steerable catheter including a helical coil of wire having a longitudinal axis, a distal end, a proximal end, and a lumen extending longitudinally from the distal end to the proximal end, a ribbon having a distal end, and extending longitudinally, adjacent to the longitudinal axis, within the lumen, from the distal end of the helical coil to the proximal end of the helical coil, and a tip formed by a laser-welded fusion of the distal end of the helical coil and the distal end of the ribbon, the tip being arranged to close the distal end of the helical coil, and operatively arranged to move in a first direction away from the longitudinal axis when the ribbon is forced in a first longitudinal direction, and move in a second direction away from the longitudinal axis when the ribbon is forced in a second longitudinal direction.

### FIELD OF THE INVENTION

The invention broadly relates to catheters. More particularly, the invention relates to coil catheters and, even more particularly to steerable coil catheters.

### BACKGROUND OF THE INVENTION

Coil catheters are well known and commonly used, particularly for epidural injections. Generally, a coil catheter comprises a stainless steel, helical coil having a lumen, and a plurality of openings near its distal end for delivering drugs into the surrounding space and tissues. Commonly, its distal end is closed via a tip assembly comprising an initially separate tip that is fixed to the distal end of the coil via some type of adhesive or welding.

One problem with prior coil catheters is that the tips have a tendency to detach from the coil while the catheter is still inserted within a patient. This can lead to whole host of medical problems, as well as product liability and medical malpractice lawsuits. Often, a tip detaches because the means for affixing it to the end of the coil is inadequate, due to the general inability to assemble and connect such small components. This problem is exacerbated when the attached tip has a diameter greater than the diameter of the coil, as the oversized tip tends to get caught on tissue, which can lead to tissue damage during operation, and on the end of the introducers used to provide the catheter access into the target tissue and epidural space.

Another problem with prior coil catheters is that they are difficult to direct after they have been inserted into the target area. Generally, a stylet comprising a wire is inserted into the lumen of the coil at the proximal end, and is used to provide some rigidity to the flexible coil and direct the tip through the tissue to the target area. A stylet is only operatively arranged to direct the tip linearly forward, and provides no means for redirecting the tip in a different direction. This drawback prevents a user from steering the catheter in and around structures, such as, spinal nerves and bone processes, which limits his ability to effectively reach target tissues he wishes to expose to drugs, such as, anti-inflammatories, steroids, analgesics, etc.

Thus, there is a long-felt need for a coil catheter having a non-detachable tip. There is also a long-felt need for a steerable coil catheter.

### BRIEF SUMMARY OF THE INVENTION

The present invention broadly is a steerable catheter comprising: a helical coil of wire, the helical coil comprising an outer diameter; an inner diameter; a longitudinal axis; a distal end; a proximal end; and, a lumen extending longitudinally from the distal end to the proximal end, the helical coil having at least a first section of loosely coiled loops defining at least one gap; a ribbon having a distal end and a proximal end, the ribbon extending longitudinally, adjacent to the longitudinal axis, within the lumen, from the distal end of the helical coil to the proximal end of the helical coil; a tip comprising a laser-welded fusion of the distal end of the helical coil and the distal end of the ribbon, the tip being arranged to close the distal end of the helical coil, and having a diameter substantially equal to or less than the outer diameter of the helical coil, wherein the tip is operatively arranged to move in a first direction away from the longitudinal axis when the ribbon is forced in a first longitudinal direction, and move in a second direction away from the longitudinal axis when the ribbon is forced in a second longitudinal direction.

The present invention also includes a method of making a steerable catheter tip comprising: providing a helical coil of wire of laser weldable material, the helical coil comprising a longitudinal axis, a distal end, a proximal end, and a lumen extending longitudinally from the distal end to the proximal end; providing a ribbon of laser weldable material having a distal end and a proximal end, the ribbon extending longitudinally, adjacent to the longitudinal axis, within the lumen, from the distal end of the helical coil past the proximal end of the helical coil; applying a first set of pulses from a laser welder to the distal end of the ribbon and the distal end of the helical coil, the first set of pulses being sufficient to generate a molten fusion of the distal end of the ribbon and the distal end of the helical coil; and, applying a second set of pulses from a laser welder to the molten fusion sufficient to shape the molten fusion into a tip arranged to close the distal end of the helical coil.

It is a general object of the present invention to provide a coil catheter having a non-detachable tip.

It is another general object of the present invention to provide a steerable coil catheter.

These and other objects and advantages of the present invention will be readily appreciable from the following description of preferred embodiments of the invention and from the accompanying drawings and claims.

### BRIEF DESCRIPTION OF THE DRAWINGS

The nature and mode of operation of the present invention will now be more fully described in the following detailed description of the invention taken with the accompanying drawing figures, in which:

FIG. 1 is a perspective view of the present invention steerable catheter;

FIG. 2 is a side plan view of a helical coil and a control ribbon of the steerable catheter, showing the first and second steps in the method of making the steerable catheter;

FIG. 3 is a side plan view of the coil and control ribbon, showing the third step in the method of making the steerable catheter;

FIG. 4 is a side plan view of the coil and control ribbon, showing the fourth step in the method of making the steerable catheter;

FIG. 5 is a side plan view of the steerable catheter in resting state;

FIG. 6 is a side plan view of the steerable catheter, showing its tip steered in a first direction; and,

FIG. 7 is a side plan view of the steerable catheter, showing its tip steered in a second direction.

### DETAILED DESCRIPTION OF THE INVENTION

At the outset, it should be appreciated that like drawing numbers on different drawing views identify identical, or functionally similar, structural elements of the invention. While the present invention is described with respect to what is presently considered to be the preferred aspects, it is to be understood that the invention as claimed is not limited to the disclosed aspects.

Furthermore, it is understood that this invention is not limited to the particular methodology, materials and modifications described and as such may, of course, vary. It is also understood that the terminology used herein is for the purpose of describing particular aspects only, and is not intended to limit the scope of the present invention, which is limited only by the appended claims.

Unless defined otherwise, all technical and scientific terms used herein have the same meaning as commonly understood to one of ordinary skill in the art to which this invention belongs. Although any methods, devices or materials similar or equivalent to those described herein can be used in the practice or testing of the invention, the preferred methods, devices, and materials are now described.

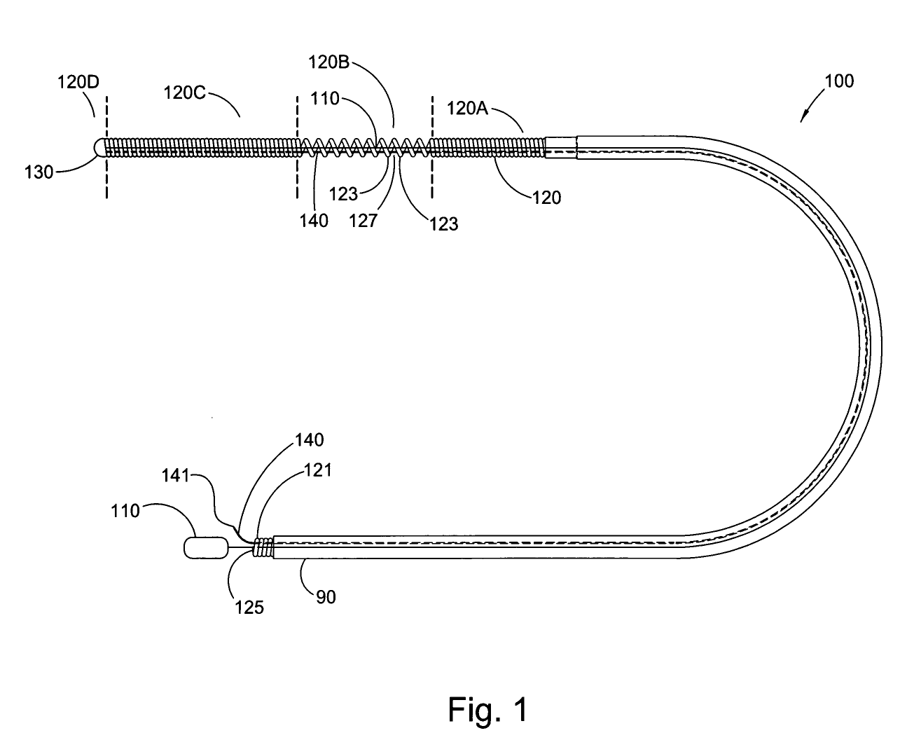

The following description of is best understood in view of FIGS. 1-7. FIG. 1 shows steerable catheter 100 broadly comprising helical coil 120, control ribbon 140, and tip 130. In preferred embodiments, steerable catheter 100 further comprises coating 90, which covers a portion of helical coil 120, and stylet 110. Stylet 110 preferably comprises a stainless steel wire and molded plastic hub, wherein the wire is insertable in one end of helical coil, and is arranged to provide the rigidity necessary to push catheter 100 through tissue.

Helical coil 120 comprises wire 126 helically arranged in series of loops 123, which are preferably substantially uniform. Helical coil 120 includes an outer diameter, an inner diameter, longitudinal axis A-A′, distal end 129, proximal end 121, and lumen 125, which extends longitudinally from distal end 129 to proximal end 121. Helical coil 120 preferably includes proximal section 120A, in which loops 123 are tightly coiled, at least a first section 120B of loosely coiled loops 123, distal section 120C, which, prior to the formation of tip 130, as described in detail infra, includes terminal section 120D. Loops 123 of section 120B define at least one, but preferably eleven to thirteen (11-13), gaps 127 which are operatively arranged to allow fluids to pass between lumen 125 and the exterior of helical coil 120. It is contemplated that helical coil 120 may comprise a plurality of loosely coiled sections, such as section 120B, in order to provide multiple drug delivery sites.

Wire 126 may comprise any material suitable for laser welding, such as, steels, nickel alloys, titanium, some aluminum alloys, and copper, but preferably comprises medical grade 304V stainless steel. Further, wire 126 comprises a substantially circular cross-section having a diameter ranging from 0.001-0.005 inches (in.), as a substantially planar, ribbon-like wire may provide insufficient material and structure when forming tip 130, as described in more detail infra. Depending on the intended catheter size, wire 126 must be the correct size within 0.0001 in. Wire 126 should be inspected for surface abrasions, as there cannot be any imperfections in the surface finish. The process of forming wire into a helical coil via an arbor having progressive feed controls, predetermined stacking parameters, spring collection means, and a support tube is well known and, therefore, not reiterated herein. Preferably, helical coil 120 has an outer diameter ranging from approximately 0.015-0.027 in., and an inner diameter ranging from 0.011-0.017 in.

Preferably, catheter 100 includes coating 90 covering at least a portion of section 120A, with a thickness ranging from 0.0005-0.002 in. A coated section of helical coil 120 preferably has a total outer diameter ranging from 0.016-0.031 in. Coating 90 may be any polymeric, heat-shrink tubing suitable for use on a catheter.

Control ribbon 140 is preferably a ribbon comprising any material suitable for laser welding, as described supra, but preferably medical grade 304V stainless steel. Control ribbon preferably has a width ranging from 0.002-0.005 in., and comprises distal end 149 and proximal end 141, and extends longitudinally, adjacent to longitudinal axis A-A′, within lumen 125, from distal end 129 to proximal end 121 of helical coil 120. In a preferred embodiment, control ribbon 140 extends past proximal end 121.

Tip 130 comprises laser-welded fusion 131 of distal end 129 of helical coil 120 and distal end 149 of control ribbon 140, tip 140 being arranged to close distal end 129. Preferably, tip 130 is substantially spherical, but may also be hemi-spherical, conical, frustoconical, etc. Further, tip 130 has a diameter substantially equal to or less than the outer diameter of helical coil 120. The method of making tip 140, which is considered an aspect of the present invention, is described in more detail infra.

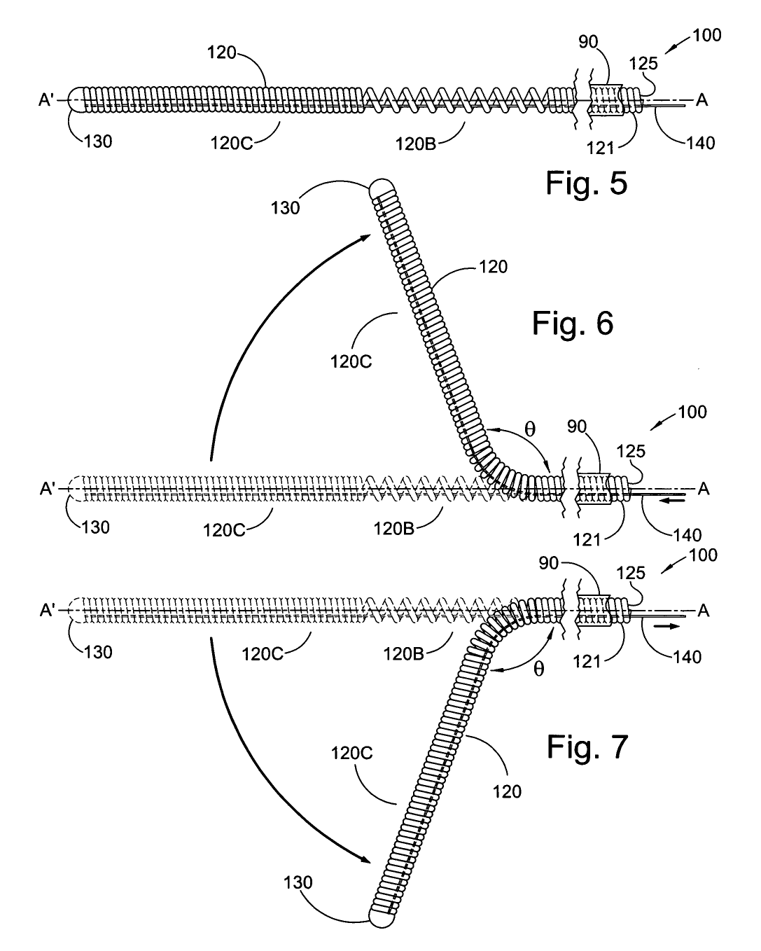

The steerability of catheter 100 is provided, in part, be the off-center arrangement of control ribbon 140 within lumen 125. Since control ribbon 140 is arranged longitudinally and adjacent to longitudinal axis A-A′, it extends from tip 130 at an off-center position. With this arrangement, tip 130 is operatively arranged to move and/or deflect in a first direction away from longitudinal axis A-A′ when control ribbon 140 is forced in a first longitudinal direction, for example, when it is forced in a direction toward tip 140, as illustrated in FIG. 6. Additionally, tip 130 is operatively arranged to move and/or deflect in a second direction away from longitudinal axis A-A′ when control ribbon 140 is forced in a second longitudinal direction, for example, when it is forced in a direction away from tip 130, as illustrated in FIG. 7. The movement and/or deflection of tip 130 away from axis A-A′ is also provided by the flexibility of helical coil 120, particularly at section 120B, wherein gaps 127 allow adjacent loops 123 to shift independently from each other. In the embodiment shown in FIGS. 5-7, helical coil 120 is arranged to bend at angle θ, which is less than or equal approximately seventy degrees (70°), when control ribbon 140 is forced in the first and/or second longitudinal direction.

Steerable catheter 100 is formed utilizing via the following hereinbelow. The method involves laser welding, preferably with a pulse Nd-YAG (neodymium-yttrium aluminum garnet) laser welder running at 33.5 watts (W). As will be readily apparent to those have skill in the relevant art, the particular power ramping, pulse repetition, shaping, and width, and laser parameters used in the method will primarily depend upon the particular materials and dimensions selected for helical coil 120 and control ribbon 140.

Regardless of the particular materials and/or dimensions selected, the method of making steerable catheter 100 comprises the following steps.

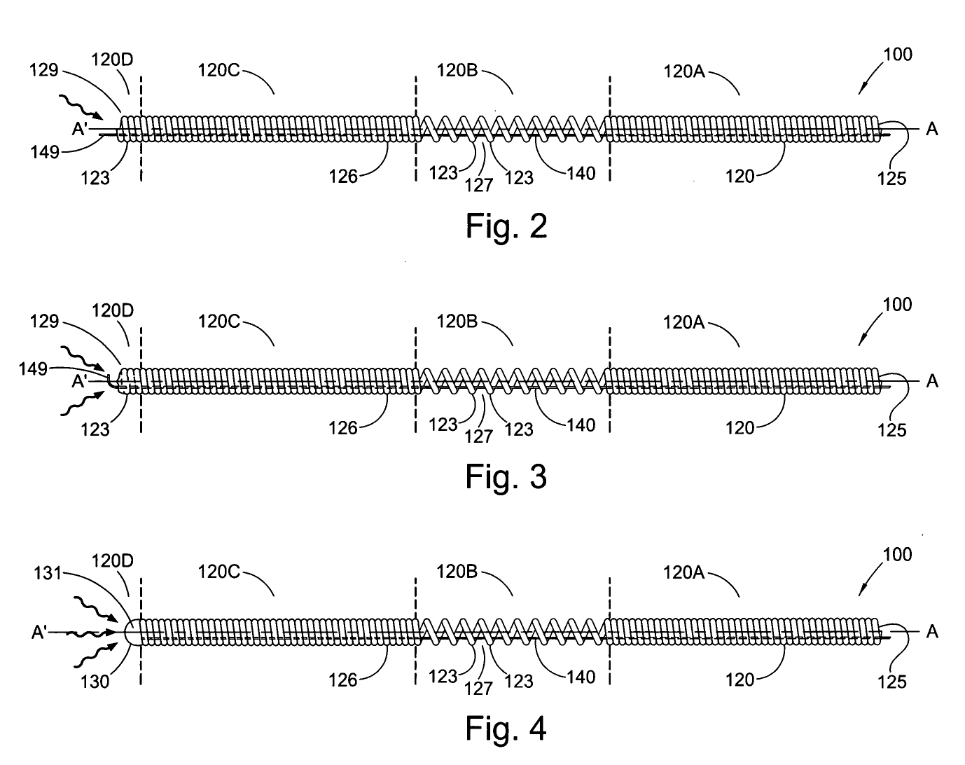

First, helical coil 120 is provided and held stationary, for example, using a fixture clamp (not shown). Second, control ribbon 140 is provided and arranged longitudinally within lumen 125 such that distal end 149 is proximate distal end 129. The first two steps are illustrated in FIG. 2.

Third, as shown in FIG. 3, the laser of the laser welder is focused on distal end 149, as depicted with the curved arrows, and applies a first set of pulses sufficient to weld distal end 149 to distal end 129. It should be readily apparent to those having skill in the art that an inert shielding gas, such as argon, should be applied to the target area of the laser in order to protect the weld from the oxygen and nitrogen present in the air.

As stated supra, the particular pulse repetition rate and pulse widths applied during this step will depend on the selected materials and dimensions of helical coil 120 and control ribbon 140. However, an important purpose of this step is to weld distal end 149 of control ribbon 140 at the previously describe off-center position, which is best accomplished by selecting a pulse repetition rate and pulse width that is sufficient to weld distal end 149 to distal end 129, while minimizing the conversion of distal end 129 to a molten state. This aspect is illustrated in Figure xx which shows distal end 149 welded to one side of distal end 129, with minimal deformation of distal end 129. This figure also shows distal end 149 curled over lumen 125 as begins to become molten. In this way, control ribbon 140 also serves as a filler rod providing the initial material needed to bridge the gap between the loops 123 of terminal section 120D.

Fourth, as shown in FIG. 4, the laser applies a second set of pulses, as depicted with the curved arrows, to the curled distal end 149 and the loops 123 of terminal section 120D. The second set of pulses should be sufficient to form a molten fusion of distal end 149 and distal end 129, including terminal section 120D. The number of coils 123 should be selected to provide enough molten material, when combined with the molten distal end 149 of control ribbon 140, to shape the fusion into tip 130, such that tip 130 is arranged to close distal end 129. The second set of pulses should also be selected to form tip 140 into a uniform shape, such as a sphere, hemisphere, cone, frustum, etc., having a diameter substantially equal to or less than the outer diameter of helical coil 120.

Tip 130 should be formed such that it is operatively arranged to move in a first direction away from longitudinal axis A-A′ when control ribbon 140 is forced in a first longitudinal direction, and to move in a second direction away from longitudinal axis A-A′ when control ribbon 140 is forced in a second longitudinal direction. Preferably, control ribbon 140 should be pull tested after tip 130 to at least 0.2 pounds per square inch (lbs/in.²).

Thus, it is seen that the objects of the present invention are efficiently obtained, although modifications and changes to the invention should be readily apparent to those having ordinary skill in the art, which modifications are intended to be within the spirit and scope of the invention as claimed. It also is understood that the foregoing description is illustrative of the present invention and should not be considered as limiting. Therefore, other embodiments of the present invention are possible without departing from the spirit and scope of the present invention.

1. A steerable catheter comprising:
a helical coil of wire, the helical coil comprising an outer diameter; an inner diameter; a longitudinal axis; a distal end; a proximal end; and, a lumen extending longitudinally from the distal end to the proximal end, the helical coil having at least a first section of loosely coiled loops defining at least one gap;
a ribbon having a distal end and a proximal end, the ribbon extending longitudinally, adjacent to the longitudinal axis, within the lumen, from the distal end of the helical coil to the proximal end of the helical coil;
a tip comprising a laser-welded fusion of the distal end of the helical coil and the distal end of the ribbon, the tip being arranged to close the distal end of the helical coil, and having a diameter substantially equal to or less than the outer diameter of the helical coil, wherein the tip is operatively arranged to move in a first direction away from the longitudinal axis when the ribbon is forced in a first longitudinal direction, and move in a second direction away from the longitudinal axis when the ribbon is forced in a second longitudinal direction.

2. The steerable catheter as recited in claim 1 wherein the helical coil is at least partially covered in a coating.

3. The steerable catheter as recited in claim 1 wherein the helical coil and the ribbon both comprise stainless steel.

4. The steerable catheter as recited in claim 1 wherein the tip is substantially spherical.

5. The steerable catheter as recited in claim 1 wherein the wire has a substantially circular cross-section.

6. A method of making a steerable catheter comprising:
providing a helical coil of wire of laser weldable material, the helical coil comprising a longitudinal axis, a distal end, a proximal end, and a lumen extending longitudinally from the distal end to the proximal end;
providing a ribbon of laser weldable material having a distal end and a proximal end, the ribbon extending longitudinally, adjacent to the longitudinal axis, within the lumen, from the distal end of the helical coil past the proximal end of the helical coil;
applying a first set of pulses from a laser welder to the distal end of the ribbon and the distal end of the helical coil, the first set of pulses being sufficient to generate a molten fusion of the distal end of the ribbon and the distal end of the helical coil; and,
applying a second set of pulses from a laser welder to the molten fusion sufficient to shape the molten fusion into a tip arranged to close the distal end of the helical coil.

7. The method recited in claim 6 wherein both the helical coil and the ribbon comprise stainless steel.

8. The method recited in claim 6 wherein the helical coil further comprises at least a first section of loosely coiled loops defining at least one gap, an outer diameter, and an inner diameter, wherein the tip has a diameter substantially equal to or less than the outer diameter of the helical coil.

9. The method recited in claim 6 wherein the helical coil is at least partially covered in a coating comprising.

10. The method recited in claim 6 wherein the tip is operatively arranged to move in a first direction away from the longitudinal axis when the ribbon is forced in a first longitudinal direction, and move in a second direction away from the longitudinal axis when the ribbon is forced in a second longitudinal direction.

11. The method recited in claim 6 wherein the laser welder is a pulse Nd-YAG micro laser welder.

12. The method catheter as recited in claim 6 wherein the tip is substantially spherical.

13. The method as recited in claim 6 wherein the wire has a substantially circular cross-section.

14. The steerable catheter made according to the method recited in claim 6.

15. A steerable catheter comprising:
a helical coil of wire, the helical coil comprising an outer diameter; an inner diameter; a longitudinal axis; a distal end; a proximal end; and, a lumen extending longitudinally from the distal end to the proximal end, the helical coil having at least a first section of loosely coiled loops defining at least one gap;
a ribbon having a distal end and a proximal end, the ribbon extending longitudinally, adjacent to the longitudinal axis, within the lumen, from the distal end of the helical coil substantially to the proximal end of the helical coil;
a tip formed by laser-welding the distal end of the helical coil and the distal end of the ribbon, the tip being arranged to close the distal end of the helical coil, and having a diameter substantially equal to or less than the outer diameter of the helical coil, wherein the tip is operatively arranged to move in a first direction away from the longitudinal axis when the ribbon is forced in a first longitudinal direction, and move in a second direction away from the longitudinal axis when the ribbon is forced in a second longitudinal direction.

16. The steerable catheter as recited in claim 15 wherein the helical coil is at least partially covered in a coating.

17. The steerable catheter as recited in claim 15 wherein the helical coil and the ribbon both comprise stainless steel.

18. The steerable catheter as recited in claim 15 wherein the tip is substantially spherical.

19. The steerable catheter as recited in claim 15 wherein the wire has a substantially circular cross-section.

20. The steerable catheter as recited in claim 15 wherein the laser-welding is performed by a Nd-YAG laser welder.
